(19) United States
(12) Patent Application Publication
(10) Pub. No.: US 2009/0182332 A1
(43) Pub. Date: Jul. 16, 2009

(54) IN-LINE ELECTROSURGICAL FORCEPS

(75) Inventors: Gary L. Long, Cincinnati, OH (US); Omar J. Vakharia, Cincinnati, OH (US); Gregory J. Bakos, Mason, OH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/014,417

(22) Filed: Jan. 15, 2008

(51) Int. Cl. A61B 18/18; A61B 18/04; A61B 17/00

(52) U.S. Cl. 606/51; 606/32; 606/207

(57) ABSTRACT

An electrosurgical apparatus, system, and method are disclosed. The apparatus, includes an elongate member defines a longitudinal opening. An elongate actuator member is slideably movable within the longitudinal opening. A proximal jaw member having a proximal portion is fixedly coupled to a distal end of the elongate flexible member. A distal jaw member has a proximal portion fixedly coupled to a distal end of the elongate actuator member. A first aperture is defined between the distal portion of the distal jaw member and the proximal portion of the distal jaw member. The distal jaw member is slideably movable relative to the proximal jaw member. The system includes a handle portion to receive a proximal end of the elongate actuator member of the apparatus. A method includes preparing the apparatus for surgery.

### BACKGROUND

Haemostasis is a procedure used for stopping the flow of blood while performing therapeutic surgical procedures. Optimizing haemostasis instruments and techniques is an ongoing concern. Whether bleeding is present or an artery is near tissue to be transected, there is always a need to prevent or stop the bleeding at the transection site. Electrosurgical haemostatic techniques employ electricity to cauterize or coagulate tissue at the transection site. Electrosurgical haemostatic instruments generally employ forceps with opposing jaws to grasp and to coagulate vessels or tissue between the jaws. Electrical energy is delivered to the vessel or tissue clamped between the jaws through electrodes formed on each jaw. Each electrode is connected to the output of an electrical generator. The forceps mechanically compress the vessel or tissue and the electrical energy applied between the electrodes seals the vessels or welds the tissue located between the electrodes.

Electrosurgical forceps can be connected to the output of various generators. Controlling the output of the generator is an effective way to seal vessels with a forceps-like device (e.g., a Ligasure® device). The output of the generator is cycled to increase and decrease the power until the vessel is sealed. This type of forceps, however, requires a dedicated generator. One method for controlling the output of a generator assists the effectiveness of the forceps in sealing arteries is provided in Kennedy J. S., Stranahan P. L., Taylor K. D., Chandler J. G., “High-burst-strength, feedback-controlled vessel sealing.” Surg. Endosc. 1998;12:876-878.

There is a need, however, for improved apparatuses and techniques for grasping and coagulating vessels or welding tissue. And, there is a need to improve the effectiveness of the forceps in sealing vessels by controlling the output of the generator with various improved techniques.

### SUMMARY

In one general aspect, the various embodiments are directed to an electrosurgical apparatus. The apparatus comprises an elongate member defining a longitudinal opening. An elongate actuator member is slideably movable within the longitudinal opening. A proximal jaw member has a proximal portion fixedly coupled to a distal end of the elongate flexible member. A distal jaw member has a proximal portion fixedly coupled to a distal end of the elongate actuator member. A first aperture is defined between the distal portion of the distal jaw member and the proximal portion of the distal jaw member. The distal jaw member is slideably movable relative to the proximal jaw member.

### FIGURES

The novel features of the various embodiments are set forth with particularity in the appended claims. The various embodiments, however, both as to organization and methods of operation, together with further advantages thereof, may best be understood by reference to the following description, taken in conjunction with the accompanying drawings as follows.

FIG. 1 illustrates one embodiment of an electrosurgical instrument.

FIG. 2 is a side perspective view of one embodiment of the in-line forceps of the electrosurgical instrument shown in FIG. 1.

FIG. 3 is a side perspective view of the in-line forceps shown in FIG. 2 with the conductive sleeve omitted to show an electrically insulative sleeve disposed within an opening defined by the conductive sleeve.

FIG. 4 is a side perspective view of the in-line forceps shown in FIG. 3 with the insulative sleeve omitted to show the underlying structures of the distal jaw member and the proximal jaw member.

FIG. 5 is a side view of the embodiment of the in-line forceps shown in FIG. 2.

FIG. 6 is a side view of the embodiment of the in-line forceps shown in FIG. 3.

FIG. 7 is a side view of the embodiment of the in-line forceps shown in FIG. 4.

FIG. 8 is a side perspective view of one embodiment of in-line forceps having a distal jaw member comprising an elongate hook member.

FIG. 9 is a side perspective view of the embodiment of the in-line forceps shown in FIG. 8 with the conductive sleeve omitted to show the electrically insulative sleeve is disposed within the conductive sleeve.

FIG. 10 is a side perspective view of the embodiment of the in-line forceps shown in FIG. 9 with the insulative sleeve omitted to show the underlying structures of the distal jaw member and the proximal jaw member.

FIG. 11 is a side view of the embodiment of the in-line forceps shown in FIG. 8.

FIG. 12 is a side view of one embodiment of the in-line forceps shown in FIG. 9.

FIG. 13 is a side view of the embodiment of the in-line forceps shown in FIG. 10.

FIG. 14 is a side perspective view of one embodiment of an in-line forceps having a distal jaw member comprising multiple portions defining multiple apertures to grasp multiple portions of a vessel.

FIG. 15 is a side perspective view of the embodiment of the in-line forceps shown in FIG. 14 with the conductive sleeve omitted to show the electrically insulative sleeve disposed within the conductive sleeve.

FIG. 16 is a side perspective view of the embodiment of the in-line forceps shown in FIG. 15 with the insulative sleeve omitted to show the underlying structures of the distal jaw member and the proximal jaw member.

FIG. 17 is a side view of the embodiment of the in-line forceps shown in FIG. 14.

FIG. 18 is a side view of the embodiment of the in-line forceps shown in FIG.15.

FIG. 19 is a side view of the embodiment of the in-line forceps shown in FIG. 16.

FIG. 20 is a graphical representation of an electrical waveform of Power (Watts) along the vertical axis as a function of Time (Seconds) along the horizontal axis.

### DESCRIPTION

The various embodiments described herein are directed to electrosurgical instruments. In various embodiments, the electrosurgical instruments comprise various embodiments of in-line forceps comprising distal and proximal jaws formed with electrodes. The distal and proximal jaws may be configured to grasp, catch, pull, hold, and/or suspend vessels or tissue and to apply a compressive force thereto. Electrical energy seals the vessels or welds the tissue sufficiently for transection. Once the vessel is sealed, it can be transected without any further bleeding from the vessel. Similarly, welding stops tissue from bleeding. As used herein the term vessel refers to a tube or duct, such as an artery or vein, to contain or convey a body fluid such as blood or some other body fluid. The term tissue refers to any structural material formed of an aggregate of cells or cell products. The terms vessel and tissue may be used interchangeably without limitation. The embodiments are not limited in this context.

The various embodiments of the electrosurgical in-line forceps may be driven with electrical energy produced by a generator. In one embodiment, the output of the generator may be controlled to generate an electrical waveform effective for sealing vessels or welding tissue in combination with compressive forces applied with the electrosurgical in-line forceps. One method for controlling the output of the generator includes interrupting the electrical power output of the generator to produce an electrical waveform with a cyclical pattern. In one embodiment, this may be implemented with a timing switching circuit connected between the output of the generator and the in-line forceps. The timing switching circuit converts a continuous electrical output from the generator to a cyclical (e.g., pulsed) output having a predetermined period set by the timer. During a first time period (e.g., a few seconds), while the electrical energy coagulates the vessel, the electrical current output of the generator decreases rapidly. In subsequent time periods, the output of the generator is pulsed based on the timing circuit. Thus, the generator produces a pulsed output current waveform. The ohmic loss due to current flow heats the vessel or tissue and subsequently coagulates the vessel or tissue. The embodiments are not limited in this context.

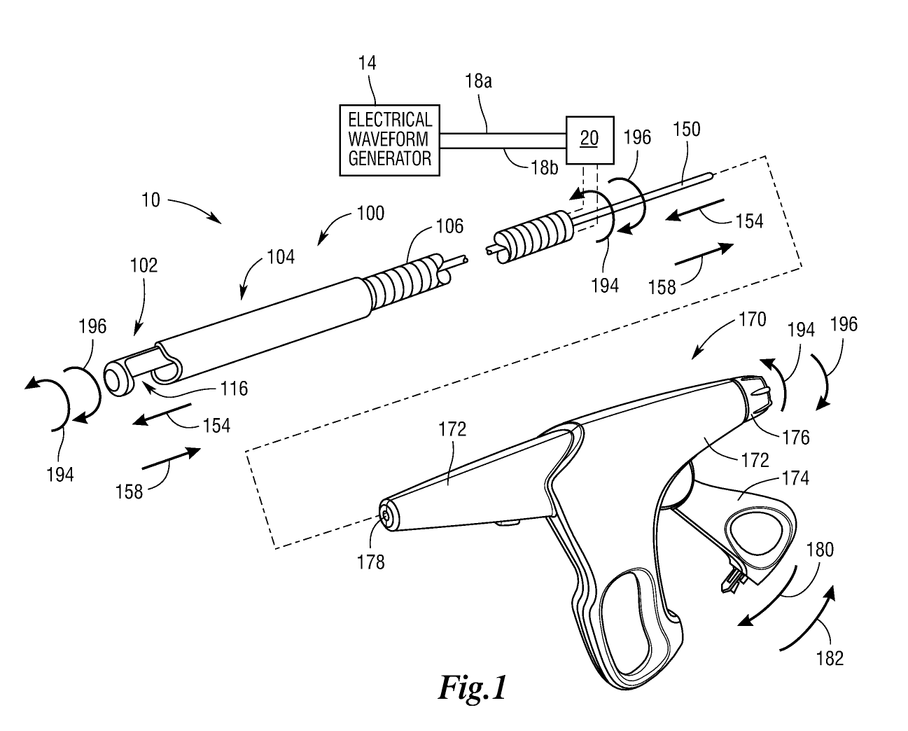

FIG. 1 illustrates one embodiment of an electrosurgical instrument 10. The electrosurgical instrument 10 may be employed to coagulate (e.g., seal) and transect (e.g., cut) vessels during surgical procedures. Similarly, the electrosurgical instrument 10 may be employed to weld tissue during surgical procedures. In one embodiment, the electrosurgical instrument 10 comprises an in-line forceps 100 and a handle assembly 170 coupled thereto. The handle assembly 170 can be manipulated by a clinician to operate the in-line forceps 100 during a surgical procedure. In one embodiment, the in-line forceps 100 comprises a distal jaw member 102 and a proximal jaw member 104. The proximal jaw member 104 is fixedly coupled to an elongate flexible member 106. The elongate flexible member 106 may be a coil pipe formed from spring steel that can be easily slideably received in a working channel of an endoscope, for example.

Using the handle assembly 170, the clinician can control the movement of the distal jaw member 102 relative to the proximal jaw member 104. The distal jaw member 102 can move reciprocally in the directions indicated by arrows 154, 158 relative to the proximal jaw member 104 along a longitudinal axis defined by an elongate actuator member 150. The elongate actuator member 150 may be substantially rigid a wire or cable to push or advance the distal jaw member 102 distally in the direction indicated by arrow 154 and, at the same time, is substantially flexible to be able to flex in conjunction with the elongate flexible member 106. The distal jaw member 102 is fixedly coupled to the elongate actuator member 150, which can move reciprocally in the directions indicated by arrows 154 and 158. Actuating the elongate actuator member 150 in the direction indicated by arrow 154 advances the distal jaw member 102 away from the proximal jaw portion 104 (e.g., opens) in the direction indicated by arrow 154 to open the distal jaw member 102. Actuating the elongate actuator member 150 in the direction indicated by arrow 158 retracts the distal jaw member 102 towards the proximal jaw member 104 (e.g., closes) in the direction indicated by arrow 158.

With the distal jaw member 102 in an open position, a vessel or tissue may be received in an aperture 116 defined between the distal jaw member 102 and the proximal jaw member 104. Actuating the elongate actuator member 150 in the direction indicated by arrow 158 actuates the distal jaw member 102 towards the proximal jaw member 104 (e.g., closes) in the direction indicated by arrow 158 to grasp the vessel located in the aperture 116. As the elongate actuator member 150 is further actuated in the direction indicated by arrow 158, the distal jaw member 102 approaches the proximal jaw member 104 to apply a compressive force to the vessel or tissue. The distal jaw member 102 and the proximal jaw member 104 forming the in-line forceps 100 cooperate to grasp, catch, pull, hold, suspend, and/or apply a compressive force to the vessel or tissue to coagulate, seal, or weld the vessel or tissue sufficiently for transection.

The distal jaw member 102 and the proximal jaw member 104 may be formed of any suitable electrically conductive materials to implement respective distal and proximal electrodes. The distal and proximal electrodes are electrically coupled to a generator 14 via respective first and second electrical conductors 18a, 18b to deliver electrical energy to the electrodes. The in-line forceps 100 may operate in bipolar or monopolar mode. Accordingly, driving the in-line forceps 100 may require a bipolar or monopolar generator. One method of controlling the output of the generator 14 includes interrupting the electrical power output to produce a cyclical pattern using a timing circuit 20 connected between the output of the generator 14 and the in-line forceps 100. The timing circuit 20 comprises suitable switching capabilities to interrupt the incoming signal and produce a cyclical or pulsed output signal to drive the in-line forceps 100. To prevent short circuiting the distal and proximal electrodes when the distal jaw member 102 is partially or fully slideably received within the proximal jaw member 104 a layer of electrical insulation is located between the distal and proximal jaw members 102, 104. The layer of electrical insulation (insulative material) electrically insulates the distal electrode from the proximal electrode when the distal jaw member 102 is slideably received within the proximal jaw member 104. The distal and proximal electrodes may comprise a relatively small surface contact area to apply a substantially high compression force (pressure) against vessels or tissue clamped between the distal jaw member 102 and the proximal jaw member 104 prior to heating the vessel with electrical energy flowing between the electrodes.

The distal and proximal jaw members 102, 104 can be implemented in various configurations. In various embodiments the distal jaw member 102 may include hook members to grasp, catch, or pull a vessel or tissue. The hook members may be relatively short or may be substantially elongate. For example, in one embodiment the distal jaw member 102 may include an elongate portion extending from a distal end of the instrument to the proximal jaw member 104 to form a hook. This feature enables the instrument to more easily grasp, catch, pull, hold, suspend, and/or apply a compressive force to a vessel to coagulate or seal the vessel sufficiently for transection grasp. In other embodiments, the distal jaw member 102 may comprise multiple portions defining multiple apertures to grasp multiple portions of a vessel. For example, a first portion of a vessel initially is received in a first aperture, then the distal jaw member 102 is pulled towards the proximal jaw member 104 and a second portion of the vessel is received in a second aperture. Additional portions of the vessel may be grasped based on the number of apertures provided, and so on, before the generator is activated to seal the vessel or tissue. This configuration and technique can be employed to seal a longer portion of the vessel or weld larger sections of tissue with minimal action. The embodiments are not limited in this context.

The handle assembly 170 may be used to operate the in-line forceps 100. In one embodiment, the handle assembly 170 comprises a base handle portion 172, a trigger 174, a rotation knob 176, and an opening 178 to receive a distal end of the elongate actuator member 150. The trigger 174 is operatively coupled to the elongate actuator member 150. When the trigger 174 is pivotally moved (e.g., squeezed) in the direction indicated by arrow 180, the elongate actuator member 150 is retracted in the direction indicated by arrow 158, and the distal jaw portion 102 closes in the direction indicated by arrow 158. When the trigger 174 is pivotally moved (e.g., released) in the direction indicated by arrow 182, the elongate actuator member 150 advances in the direction indicated by arrow 154, and the distal jaw portion 102 opens in the direction indicated by arrow 154. The proximal end of the elongate actuator member 150 is fixedly received within a neck portion of the rotation knob 176. When the rotation knob 176 is rotated in the direction indicated by arrow 194 the elongate actuator member 150 and the distal jaw portion 102 also rotate in the direction indicated by arrow 194. When the rotation knob 176 is rotated in the direction indicated by arrow 196 the elongate actuator member 150 and the distal jaw portion 102 also rotate in the direction indicated by arrow 196. The embodiments are not limited in this context.

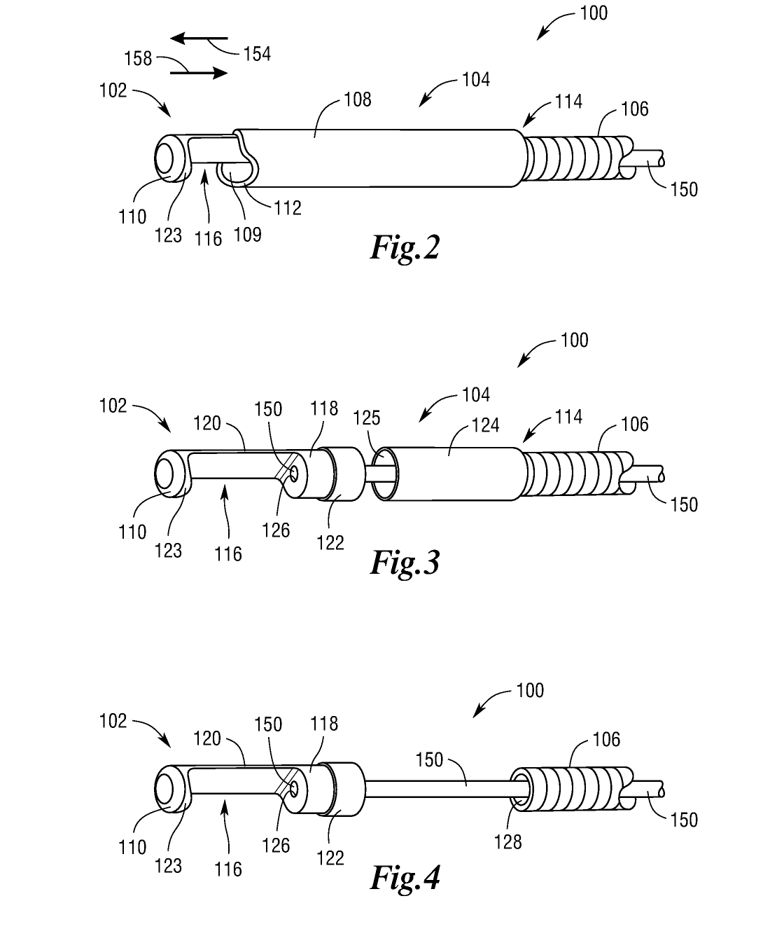

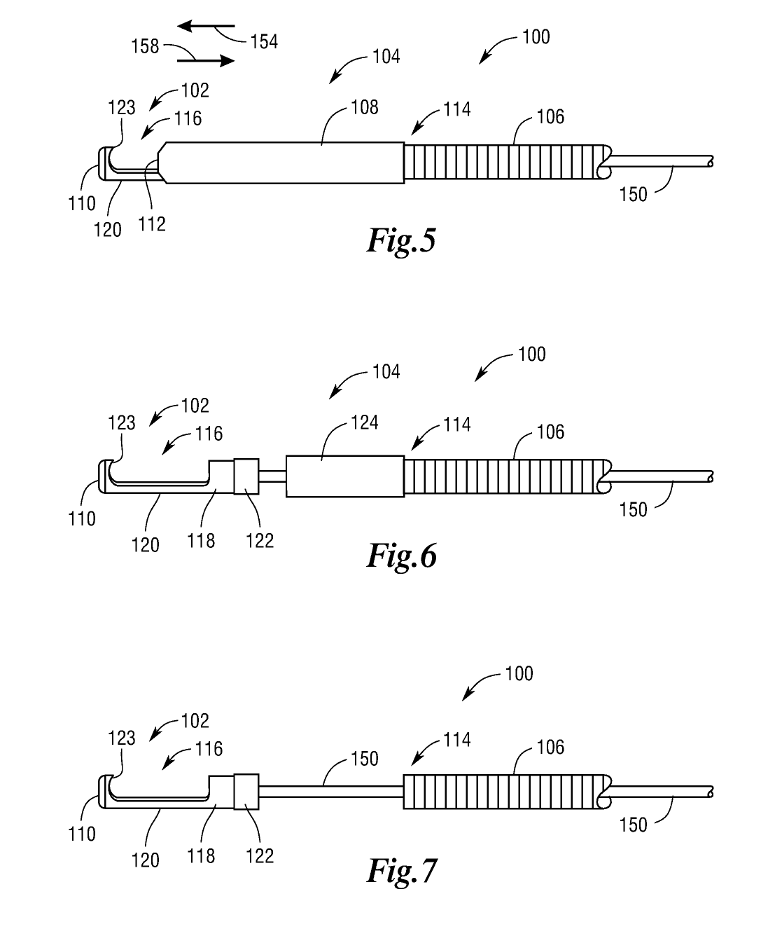

FIG. 2 is a side perspective view of one embodiment of the in-line forceps 100 of the electrosurgical instrument 10 shown in FIG. 1. FIG. 5 is a side view of the embodiment of the in-line forceps 100 shown in FIG. 2. Referring now to FIGS. 2 and 5, in one embodiment, the distal jaw member 102 is formed of any suitable electrically conductive material (e.g., brass, stainless steel) and is referred to herein as a distal electrode. The proximal jaw member 104 comprises an electrically conductive sleeve 108 defining an opening 109 therethrough. The electrically conductive sleeve 108 is formed of any suitable electrically conductive material (e.g., brass, stainless steel) and is referred to herein as a proximal electrode. A hook member 123 projects proximally from a first portion 110 of the distal jaw member 102. The hook member 123 is employed to grasp a vessel or tissue. The conductive sleeve 108 comprises a first portion 112. The first portion 110 of the distal jaw member 102 and the first portion 112 of the conductive sleeve 108 are configured to apply a suitable compressive force against a vessel or tissue located therebetween in response to actuating the handle assembly. Once the vessel or tissue is clamped, energy in the form of a predetermined electrical waveform is delivered to the clamped vessel or tissue by the electrical waveform generator 14 to coagulate and transect the vessel or weld the tissue. A second portion 114 of the conductive sleeve 108 is fixedly coupled to the elongate flexible member 106. Thus, the conductive sleeve 108 is fixed relative to the distal jaw member 102.

FIG. 3 is a side perspective view of the embodiment of the in-line forceps 100 shown in FIG. 2 with the conductive sleeve 108 omitted to show an electrically insulative sleeve 124 disposed within the opening 109 defined by the conductive sleeve 108. The electrically insulative sleeve 124 defines an opening 125 therethrough. FIG. 6 is a side view of the embodiment of the in-line forceps 100 shown in FIG. 3. Referring now to FIGS. 3 and 6, the first portion 110 of the distal jaw member 102 is located at a distal end thereof and a second portion 118 is located at a proximal end thereof. The second portion 118 of the distal jaw member 102 is fixedly coupled to a distal end of the elongate actuator member 150. In the illustrated embodiment, the second portion 118 defines an opening 126 to receive the distal end of the elongate actuator member 150. The distal end of the elongate actuator member 150 may be fixedly coupled to the second portion 118 by any suitable means, such as friction, crimp, weld, solder, screw, and the like. The second portion 118 is configured to be slideably received within the opening defined by the electrically insulative sleeve 124 is disposed within the opening 125 defined by the conductive sleeve 108. Thus, the distal electrode (e.g., the distal jaw member 102) is electrically insulated from the proximal electrode (e.g., the proximal jaw member 104). Accordingly, when the distal electrode is retracted within the proximal electrode in the direction indicated by arrow 158, the two electrodes are electrically isolated from each other. The electrically insulative sleeve 124 is formed of a substantially frictionless (e.g., lubricious) material. Thus, the second portion 118 is easily slideably received within the insulative sleeve 124. To further decrease any friction between the distal jaw member 102 and the insulative sleeve 124, an electrically insulative bushing 122 is coupled to a distal end of the elongate actuator member 150 and located adjacent to the second portion 118 of the distal jaw member 102. The electrically insulative bushing 122 is formed of a substantially frictionless (e.g., lubricious) material. The electrically insulative bushing 122 and the insulative sleeve 124 may be fabricated from polyimide TEFLON® materials, which provide a substantially lubricious surface and are good electrical insulators. Accordingly, as the distal jaw member 102 is retracted in the direction indicated by arrow 158, the bushing 122 and the second and third portions 118, 120 of the distal jaw member 102 are easily slideably received within the insulative sleeve 124. A third portion 120 of the distal jaw member 102 is formed intermediate the first and second portions 110, 118. The first, second, and third portions 110, 118, 120, and the hook member 123 define the aperture 116 for receiving a vessel or tissue therein.

FIG. 4 is a side perspective view of the embodiment of the in-line forceps 100 shown in FIG. 3 with the insulative sleeve 124 omitted to show the underlying structures of the distal jaw member 102 and the proximal jaw member 104. FIG. 7 is a side view of the embodiment of the in-line forceps 100 shown in FIG. 4. Referring now to FIGS. 4 and 7, the elongate actuator member 150 is slideably received within a longitudinal opening 128 formed within the elongate flexible member 106. The elongate actuator member 150 is slideably movable within the longitudinal opening 128 in response to actuating the hand assembly 170.

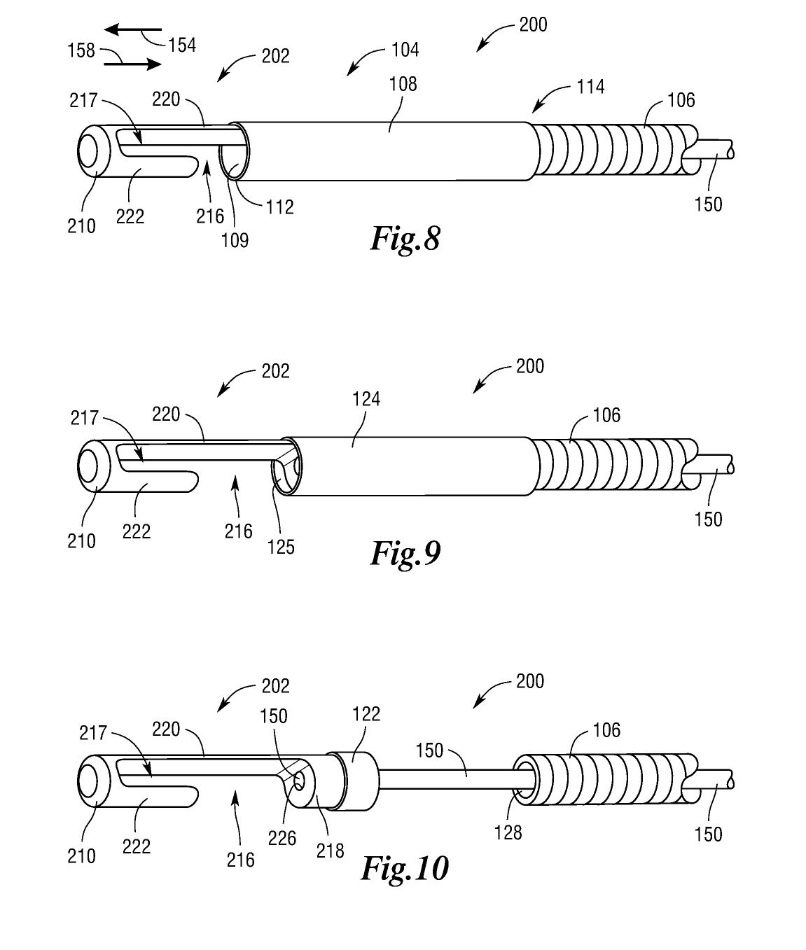

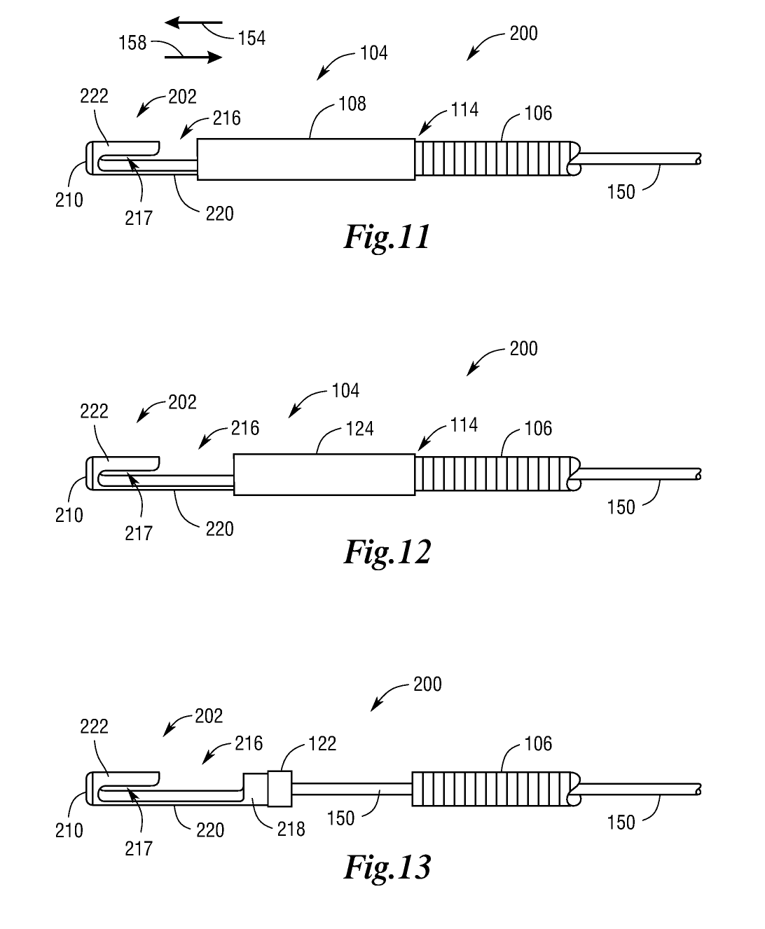

FIG. 8 is a side perspective view of one embodiment of in-line forceps 200 having a distal jaw member 202 comprising an elongate hook member 222. The proximal jaw member 104, the elongate flexible member 106, and the elongate actuator member 150 are similar to those discussed above with reference to FIGS. 1-7 and for succinctness the description is not repeated. FIG. 11 is a side view of the embodiment of the in-line forceps 200 shown in FIG. 8. FIG. 9 is a side perspective view of the embodiment of the in-line forceps 200 shown in FIG. 8 with the conductive sleeve 108 omitted to show the electrically insulative sleeve 124 is disposed within the conductive sleeve 108. FIG. 12 is a side view of one embodiment of the in-line forceps 200 shown in FIG. 9. FIG. 10 is a side perspective view of the embodiment of the in-line forceps 200 shown in FIG. 9 with the insulative sleeve 124 omitted to show the underlying structures of the distal jaw member 202 and the proximal jaw member 104. FIG. 13 is a side view of the embodiment of the in-line forceps 200 shown in FIG. 10.

Referring now to FIGS. 8-13, in one embodiment, the distal jaw member 202 electrode (e.g., distal electrode) may be formed of any suitable electrically conductive material (e.g., brass, stainless steel). The elongate hook member 222 extends proximally from the first distal portion 210 of the distal jaw member 202. A first aperture 216 is defined at the proximal end of the distal jaw member 102 to receive a vessel or tissue therein. A second aperture 218 is defined by the elongate hook member 222 to grasp, catch, pull, hold, and/or suspend the vessel or tissue received within the first aperture 216.

The first portion 210 is located at a distal end of the distal jaw member 202 and a second portion 218 is located at a proximal end of the distal jaw member 202. The second portion 218 of the distal jaw member 202 is fixedly coupled to the distal end of the elongate actuator member 150. In the illustrated embodiment, the second portion 218 defines an opening 226 to receive the distal end of the elongate actuator member 150 by any suitable means such as friction, crimp, weld, solder, screw, and the like. The second portion 218 is slideably received within the electrically insulative sleeve 124 disposed within the conductive sleeve 108. The insulative sleeve 124 electrically insulates the distal jaw member 202 (e.g., distal electrode) from the proximal jaw member 104 (e.g., proximal electrode). As previously described, the electrically insulative sleeve 124 is formed of substantially frictionless (e.g., lubricious) material. Thus, the second portion 218 is easily slideably received within the insulative sleeve 124. As previously discussed, to further decrease any friction between the distal jaw member 202 and the insulative sleeve 124, the substantially frictionless (e.g., lubricious) electrically insulative bushing 122 is fixedly coupled to the second portion 218 of the distal jaw member 202. Accordingly, as the distal jaw member 202 is retracted in the direction indicated by arrow 158, the bushing 122 and the proximal portion of the distal jaw member 102 are easily slideably received within the insulative sleeve 124 with minimal frictional resistance. The third portion 220 is formed intermediate the first and second portions 210, 218. The first aperture 216 is defined by the proximal end of the elongate hook member 222, and the second and third portions 210, 218, 220. The second aperture 218 is defined by the first portion 210, the third portion 220, and the elongate hook member 222. The elongate actuator member 150 is easily slideably received within a longitudinal opening 128 formed within the elongate flexible member 106.

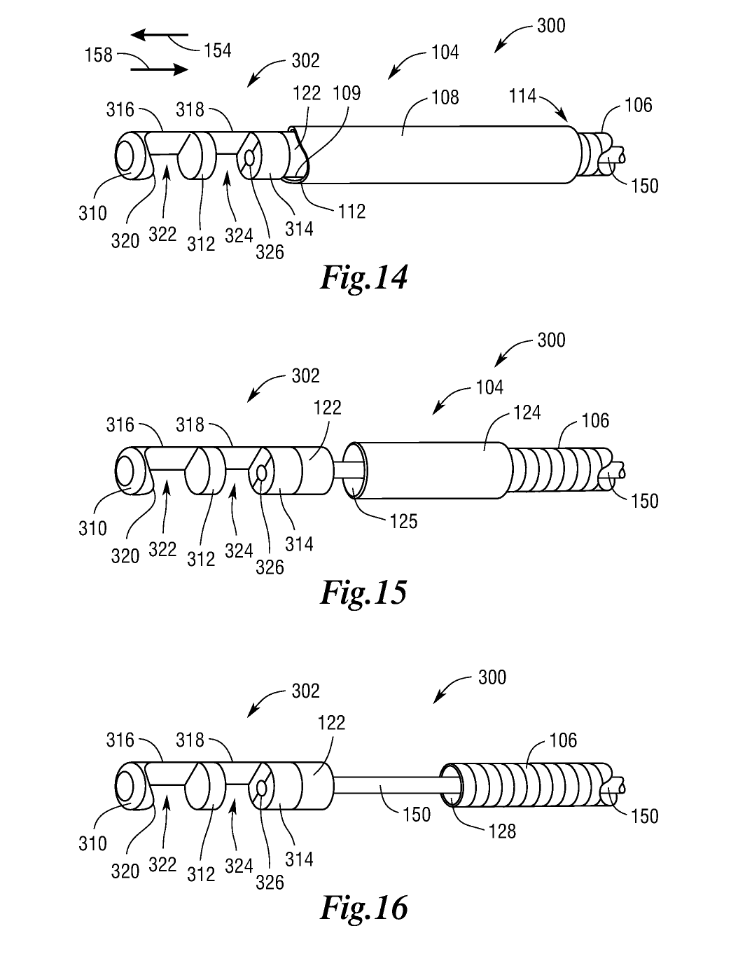

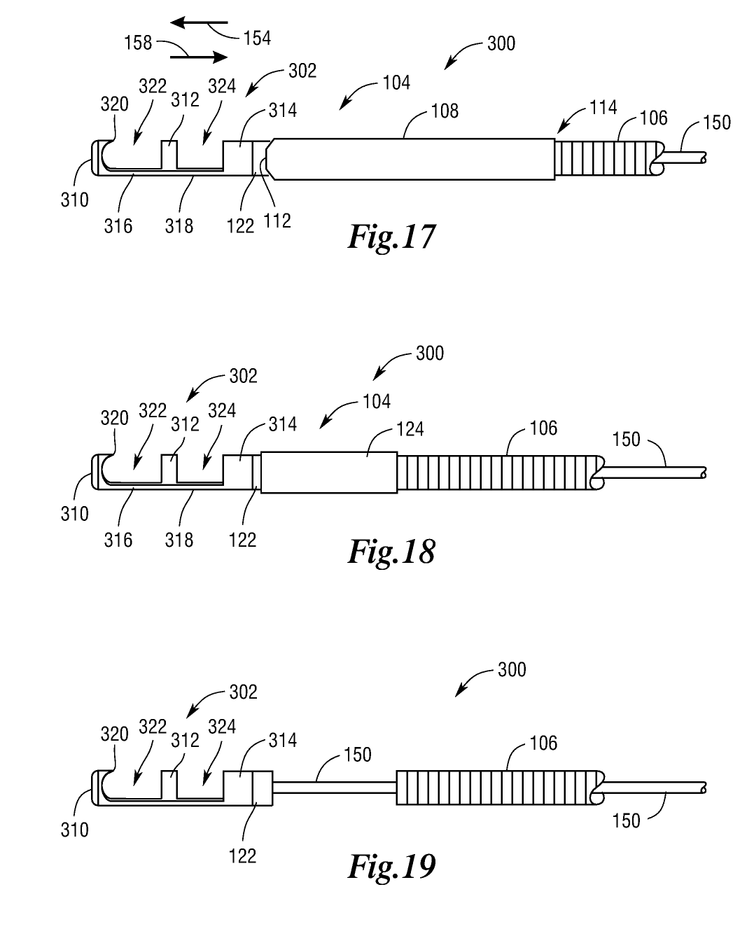

FIG. 14 is a side perspective view of one embodiment of an in-line forceps 300 having a distal jaw member 302 comprising multiple portions defining multiple apertures to grasp multiple portions of a vessel or tissue. The proximal jaw member 104, the elongate flexible member 106, and the elongate actuator member 150 are similar to those discussed above with reference to FIGS. 1-7 and the description for succinctness will not be repeated. FIG. 17 is a side view of the embodiment of the in-line forceps 300 shown in FIG. 14. FIG. 15 is a side perspective view of the embodiment of the in-line forceps 300 shown in FIG. 14 with the conductive sleeve 108 omitted to show the electrically insulative sleeve 124 disposed within the conductive sleeve 108. FIG. 18 is a side view of the embodiment of the in-line forceps 300 shown in FIG. 15. FIG. 16 is a side perspective view of the embodiment of the in-line forceps 300 shown in FIG. 15 with the insulative sleeve 124 omitted to show the underlying structures of the distal jaw member 302 and the proximal jaw member 104. FIG. 19 is a side view of the embodiment of the in-line forceps 300 shown in FIG. 16.

Referring now to FIGS. 14-19, in one embodiment, the distal jaw member 302 electrode (e.g., distal electrode) may be formed of any suitable electrically conductive material (e.g., brass, stainless steel). The distal jaw member 302 comprises a first portion 310 that defines a hook member 320 to grasp, catch, pull, hold, and/or suspend a vessel or tissue. A second portion 312 is located intermediate the first portion 310 and a third portion 314. A fourth portion 316 extends between the first portion and the second portion 312 and defines a first aperture 322. A fifth portion 318 extends between the second portion 312 and the third portion 314 and defines a second aperture 324. A first portion of a vessel initially may be received in the second aperture 324. The distal jaw member 302 is then partially retracted in the direction indicated by arrow 158 into the insulative sleeve 124 until the first portion of the vessel is clamped between the second portion 312 of the distal jaw member 302 and the first portion 112 of the proximal jaw member 104. When the first portion of the vessel is compressed between the second portion 312 of the distal jaw member 302 and the first portion 112 of the proximal jaw member 104, the generator may be activated to energize the first portion of the vessel. Subsequently, a second portion of the vessel may be received within the first aperture 322. The distal jaw member 302 is then fully retracted until the second portion of the vessel is clamped between the first portion 310 of the distal jaw member 302 and the first portion 112 of the proximal jaw member 104. When the first portion of the vessel is compressed between the first portion 310 of the distal jaw member 302 and the first portion 112 of the proximal jaw member 104, the generator may be activated to energize the second portion of the vessel. In this manner, the in-lie forceps 300 can treat a longer section of a vessel relative to sections of vessels that can be treated using the in-line forceps 100, 200. A similar procedure may be applied to weld multiple sections of tissue.

The first portion 310 is located at a distal end of the distal jaw member 302 and the third portion is located at a proximal end thereof. The third portion 314 of the distal jaw member 302 is configured to fixedly couple to the elongate actuator member 150. In the illustrated embodiment the second portion 312 is located between the first portion 310 and the third portion 318 at an intermediate distance to define two substantially equal apertures 322, 324. In other embodiments, the second portion 312 may be located anywhere between the first portion 310 and the third portion 314 to define different sized apertures. In the illustrated embodiment, the third portion defines an opening 326 to receive the elongate actuator member 150. The distal end of the elongate actuator member 150 may be fixedly coupled to the third portion 314 by any suitable means, such as friction, crimp, weld, solder, screw, and the like. The second and third portions 312, 314 are configured to be slideably received within the electrically insulative sleeve 124 disposed within the conductive sleeve 108. The insulative sleeve 124 electrically insulates the distal jaw member 320 (e.g., distal electrode) from the proximal jaw member 104 (e.g., proximal electrode). As previously described, the electrically insulative sleeve 124 is formed of substantially frictionless (e.g., lubricious) material. Thus, the second portion 218 is easily slideably received within the insulative sleeve 124. As previously discussed, to further decrease any friction between the distal jaw member 302 and the insulative sleeve 124, an electrically insulative bushing 122 substantially frictionless (e.g., lubricious) is fixedly coupled to the third portion 314 of the distal jaw member 302. The electrically insulative bushing 122 and the insulative sleeve 124 may be fabricated from polyimide TEFLON® materials. Accordingly, as the distal jaw member 302 is retracted in the direction indicated by arrow 158, the bushing 122 and the proximal portion of the distal jaw member 302 are easily slideably received within the insulative sleeve 124. The elongate actuator member 150 is slideably received within a longitudinal opening 128 formed within the elongate flexible member 106.

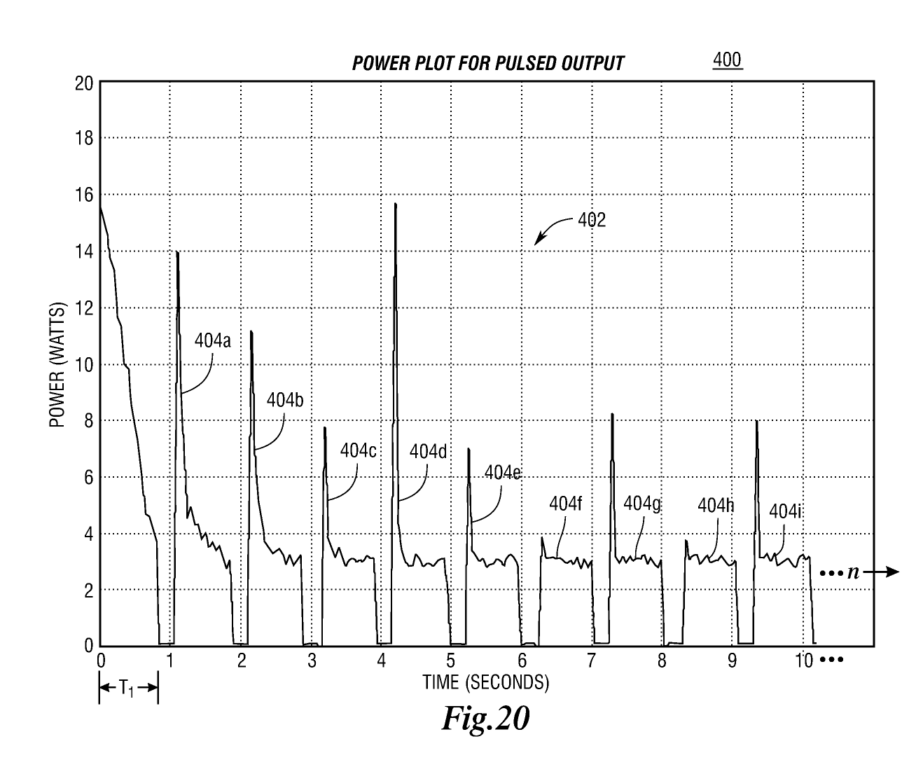

FIG. 20 is a graphical representation of an electrical waveform 400 of Power (Watts) along the vertical axis as a function of Time (Seconds) along the horizontal axis. The various embodiments of the electrosurgical in-line forceps 100, 200, 300 may be driven with electrical energy produced by the generator 14. However, for succinctness, the following description will be limited to the electrosurgical instrument 10 comprising the in-line forceps 100. Accordingly, with reference now to FIGS. 1 and 20, in one embodiment, the output of the generator 14 may be controlled to generate an electrical waveform 402 effective to seal vessels or weld tissue in combination with compressive forces applied to the vessel or tissue by the electrosurgical in-line forceps 100. One method of controlling the output of the generator 14 includes interrupting the electrical power output in a cyclical pattern using the timing circuit 20 connected between the output of the generator 14 and the in-line forceps 100. Other suitable methods for switching the output of the generator 14 may be employed without limitation. During a first time period T₁ (e.g., a few seconds), while the electrical energy coagulates the vessel, the electrical current decreases rapidly. Beyond the first time period T₁, the output of the generator 14 is pulsed to produce a series of pulses 404a-i, up to n pulses, in the current output that are suitable to seal and transect vessels and/or tissue. The ohmic loss due to current flow heats the vessel or tissue and subsequently coagulates the vessel or tissue. This may be illustrated graphically as the electrical waveform 400 in terms of Power along the vertical axis versus Time along the horizontal axis. The embodiments are not limited in this context.

In one embodiment, the distal jaw member 102 and the proximal jaw member 104 of the in-line forceps 100 are adapted to receive electrical energy from the generator 14 in the cyclical pattern illustrated in the graphical representation of the waveform 400. The electrical energy is conducted through the first and second electrical conductors 18a, 18b to the timing circuit 20, which applies the cyclic pattern and generates the waveform 400. The energy is delivered to the distal electrode (e.g., the distal jaw member 102) and the proximal electrode (e.g., the proximal jaw member) forms an electrical field between the distal and proximal electrodes suitable to seal or coagulate vessels or weld tissue. In one embodiment, the electrical waveform generator 14 may be configured to generate electrical fields at a predetermined frequency, amplitude, polarity, and pulse width suitable to seal vessels or weld tissue. The embodiments, however, are not limited in this context.

In one embodiment, the distal and proximal electrodes formed on the respective distal jaw member 102 and the proximal jaw member 104 are adapted to receive electrical fields in the form of the waveform 402 produced by the generator 14. In another embodiment, the distal and proximal electrodes are adapted to receive a radio frequency (RF) waveform from an RF generator. In one embodiment, the electrical waveform generator 14 may be a conventional, bipolar/monopolar electrosurgical generator such as one of many models commercially available, including Model Number ECM 830, available from BTX Molecular Delivery Systems Boston, Mass. The generator 14 generates electrical waveforms having predetermined frequency, amplitude, and pulse width. The application of these electrical waveforms seals or welds vessels or tissue clamped between the distal jaw member 102 and the proximal jaw member 104. Suitable electrical waveforms 402 include direct current (DC) electrical pulses delivered at a frequency in the range of 1-20 Hz, amplitude in the range of +100 to +1000 VDC, and pulse width in the range of 0.01-100 ms. For example, an electrical waveform having amplitude of +500 VDC and pulse duration of 20 ms may be delivered at a pulse repetition rate or frequency of 10 HZ to seal weld vessels or tissue.

The polarity of the distal and proximal electrodes may be switched electronically to reverse the polarity of the in-line forceps 100. In one embodiment, the polarity of the electrical pulses may be inverted or reversed by the electrical waveform generator 14. For example, the electrical pulses initially delivered at a frequency in the range of 1-20 Hz and amplitude in the range of +100 to +1000 VDC, and pulse width in the range of 0.01-100 ms. The polarity of the electrical pulses then may be reversed such that the pulses have amplitude in the range of −100 to −1000 VDC. For example, an electrical waveform comprising DC pulses having amplitude of +500 VDC may be initially applied to the treatment region or target site and after a predetermined period, the amplitude of the DC pulses may be reversed to −500 VDC. The embodiments are not limited in this context.

In one embodiment, the electrical waveform generator 14 may comprise a RF waveform generator. The RF generator may be a conventional, bipolar/monopolar electrosurgical generator such as one of many models commercially available, including Model Number ICC 350, available from Erbe, GmbH. Either a bipolar mode or monopolar mode may be used. When using the bipolar mode with two electrodes (e.g., the distal and proximal electrodes formed by the respective distal jaw member 102 and the proximal jaw member 104), one electrode is electrically connected to one bipolar polarity, and the other electrode is electrically connected to the opposite bipolar polarity. If more than two electrodes are used, the polarity of the electrodes may be alternated so that any two adjacent electrodes have opposite polarities. Either the bipolar mode or the monopolar mode may be used with the illustrated embodiment of the electrosurgical system 10. In the bipolar mode, for example, the distal electrode may be electrically connected to one bipolar polarity, and the proximal electrode may be electrically connected to the opposite bipolar polarity (or vice-versa). If more than two electrodes are used, the polarity of the distal and proximal electrodes is alternated so that any two adjacent electrodes have opposite polarities.

In either case, the electrical waveform generator 14, when using the monopolar mode with two or more electrodes, a grounding pad is not needed on the patient. Because a generator will typically be constructed to operate upon sensing connection of ground pad to the patient when in monopolar mode, it can be useful to provide an impedance circuit to simulate the connection of a ground pad to the patient. Accordingly, when the electrosurgical instrument 10 is used in monopolar mode without a grounding pad, an impedance circuit can be assembled by one skilled in the art, and electrically connected in series with either one of the distal or proximal electrodes that would otherwise be used with a grounding pad attached to a patient during monopolar electrosurgery. Use of an impedance circuit allows use of the generator 14 in monopolar mode without use of a grounding pad attached to the patient.

It will be appreciated that the terms “proximal” and “distal” are used herein with reference to a clinician gripping the handle assembly 170. Thus, the distal portion 102 is distal with respect to the more proximal handle assembly 170. It will be further appreciated that, for convenience and clarity, spatial terms such as “top” and “bottom” also are used herein with respect to the clinician gripping the handle assembly 170. However, surgical instruments are used in many orientations and positions, and these terms are not intended to be limiting and absolute.

Having described various embodiments of the electrosurgical instrument 10 comprising various embodiments of in-line bipolar forceps 100, 200, 300 to seal and transect vessels, it will be appreciated that the in-line bipolar forceps 100, 200, 300 may be inserted in a patient during a minimally invasive surgical procedure through an endoscope, laparoscope, thoracoscope, or in open surgical procedures, via small incisions or keyholes as well as other external non-invasive medical procedures. Additional electrodes may be introduced in the tissue treatment region by way of a natural orifice through a cannula or catheter. The placement and location of the in-line bipolar forceps electrodes can be important for effective and efficient therapy. Once positioned, the in-line bipolar forceps therapy electrodes are adapted to deliver electrical current to coagulate (e.g., seal) the vessel sufficiently such that it can be transected. The electrical current is generated by a control unit or generator located external to the patient. The electrical current may be characterized by a particular waveform in terms of frequency, amplitude, and pulse width.

Endoscopy refers to looking inside the human body for medical reasons. Endoscopy may be performed using an instrument called an endoscope. Endoscopy is a minimally invasive diagnostic medical procedure used to evaluate the interior surfaces of an organ by inserting a small tube into the body, often, but not necessarily, through a natural body opening or through a relatively small incision. Through the endoscope, an operator may observe surface conditions of the organs including abnormal or diseased tissue such as lesions and other surface conditions. The endoscope may have a rigid or a flexible tube and in addition to providing an image for visual inspection and photography, the endoscope may be adapted and configured for taking biopsies, retrieving foreign objects, and introducing medical instruments to a tissue treatment region referred to as the target site. Endoscopy is a vehicle for minimally invasive surgery.

Laparoscopic surgery, is a minimally invasive surgical technique in which operations in the abdomen are performed through small incisions (usually 0.5-1.5 cm), keyholes, as compared to larger incisions needed in traditional surgical procedures. Laparoscopic surgery includes operations within the abdominal or pelvic cavities, whereas keyhole surgery performed on the thoracic or chest cavity is called thoracoscopic surgery. Laparoscopic and thoracoscopic surgery belong to the broader field of endoscopy.

A key element in laparoscopic surgery is the use of a laparoscope: a telescopic rod lens system, usually connected to a video camera (single chip or three chip). Also attached is a fiber optic cable system connected to a “cold” light source (halogen or xenon), to illuminate the operative field, inserted through a 5 mm or 10 mm cannula to view the operative field. The abdomen is usually insufflated with carbon dioxide gas to create a working and viewing space. The abdomen is essentially blown up like a balloon (insufflated), elevating the abdominal wall above the internal organs like a dome. Carbon dioxide gas is used because it is common to the human body and can be removed by the respiratory system if it is absorbed through tissue.

The embodiments of electrosurgical instruments comprising in-line bipolar forceps and techniques described herein may be employed to coagulate and transect vessels. These instruments may be adapted for use in minimally invasive surgeries where they can be introduced into the patient using a trocar. The electrosurgical instruments also may be introduced into the patient endoscopically (e.g., laparoscopically and/or thoracoscopically) or through small minimally invasive incisions (e.g., keyholes). Embodiments of the electrosurgical instruments may be introduced into the patient through a natural opening of the patient are known as Natural Orifice Translumenal Endoscopic Surgery (NOTES)™.

Various embodiments of the electrosurgical instrument 10 described herein may be adapted for use in minimally invasive surgical procedures. These procedures include endoscopic, laparoscopic, thoracoscopic, or open surgical procedures via small incisions or keyholes as well as external and non-invasive medical procedures. The electrosurgical instrument 10 may be adapted for NOTES™ procedures where the instrument 10 can be positioned within a natural opening of the patient such as the colon or the esophagus and can be passed through the natural opening to reach the target site. The electrosurgical instrument 10 also may be configured to be positioned through a small incision or keyhole on the patient and can be passed through the incision to reach a target site through a trocar. Once positioned at the target site, the electrosurgical instrument 10 can be configured to coagulate and transect vessels by applying electrical energy to electrodes of the instruments 10.

In one embodiment, the electrosurgical instrument system 10 may be employed in conjunction with a flexible endoscope (also referred to as endoscope), such as the GIF-100 model available from Olympus Corporation. The flexible endoscope, laparoscope, or thoracoscope may be introduced into the patient trans-anally through the colon, the abdomen via an incision or keyhole and a trocar, or through the esophagus. The endoscope or laparoscope assists the surgeon to guide and position the electrosurgical instrument 10 near the tissue treatment region to treat diseased tissue on organs such as the liver. In another embodiment, the flexible endoscope or thoracoscope may be introduced into the patient orally through the esophagus to assist the surgeon guide and position the electrosurgical instrument 10 near the target site.

The flexible endoscope comprises an endoscope handle and an elongate relatively flexible shaft. The distal end of the flexible shaft of the flexible endoscope may comprise a light source a viewing port, and an optional working channel. The viewing port transmits an image within its field of view to an optical device such as a charge coupled device (CCD) camera within the flexible endoscope so that an operator may view the image on a display monitor (not shown).

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, said to be incorporated by reference herein, but which conflicts with existing definitions, statements, or other disclosure material set forth herein will only be incorporated to the extent that no conflict arises between that incorporated material and the existing disclosure material.

What is claimed is:

1. An electrosurgical apparatus, comprising:
an elongate member defining a longitudinal opening;
an elongate actuator member slideably movable within the longitudinal opening;
a proximal jaw member having a proximal portion fixedly coupled to a distal end of the elongate flexible member; and
a distal jaw member having a proximal portion fixedly coupled to a distal end of the elongate actuator member, a first aperture is defined between the distal portion of the distal jaw member and the proximal portion of the distal jaw member, wherein the distal jaw member is slideably movable relative to the proximal jaw member.

2. The electrosurgical apparatus of claim 1, wherein the distal jaw member and the proximal jaw member form respective distal and proximal electrodes adapted to couple to an electrical waveform generator and to receive an electrical waveform sufficient to electrically seal a vessel or weld tissue located within the first aperture.

3. The electrosurgical apparatus of claim 2, wherein the electrical waveform generator produces a pulsed energy waveform.

4. The electrosurgical apparatus of claim 1, wherein the distal portion of the distal jaw member comprises a hook member.

5. The electrosurgical apparatus of claim 4, wherein the distal portion of the distal jaw member comprises an elongate hook member that extends from the distal portion of the distal jaw member and defines a second aperture.

6. The electrosurgical apparatus of claim 1, comprising an intermediate portion located between the distal portion and the proximal portion of the distal jaw member, wherein the first aperture is defined between the distal portion and the intermediate portion of the distal jaw member and a second aperture is defined between the intermediate portion and the proximal portion of the distal jaw member.

7. The electrosurgical apparatus of claim 6, comprising a plurality of intermediate portions located between the distal portion and the proximal portion of the distal jaw member, wherein a plurality of apertures are defined between the distal portion and the proximal portion of the distal jaw member.

8. The electrosurgical apparatus of claim 1, wherein the proximal jaw member comprises an electrically conductive sleeve defining an opening therethrough.

9. The electrosurgical apparatus of claim 8, comprising an electrically insulative sleeve located within an opening defined by the conductive sleeve.

10. The electrosurgical apparatus of claim 8, comprising an electrically insulative bushing fixedly coupled to the distal end of the elongate actuator member and located adjacent to the proximal portion of the distal jaw member.

11. A electrosurgical system, comprising:
an elongate member defining a longitudinal opening;
an elongate actuator member slideably movable within the longitudinal opening;
a proximal jaw member having a proximal portion fixedly coupled to a distal end of the elongate flexible member;
a distal jaw member having a proximal portion fixedly coupled to a distal end of the elongate actuator member, a first aperture is defined between the distal portion of the distal jaw member and the proximal portion of the distal jaw member, wherein the distal jaw member is slideably movable relative to the proximal jaw member; and
a handle portion to receive a proximal end of the elongate actuator member.

12. The electrosurgical system of claim 11, comprising a generator coupled to the distal jaw member and the proximal jaw member, forming respective distal and proximal electrodes, to couple to an electrical waveform produced by the generator sufficient to electrically seal a vessel or weld tissue located within the first aperture.

13. The electrosurgical system of claim 12, comprising a timing circuit coupled between an output of the generator and the distal and proximal jaw members to produce a pulsed energy waveform.

14. The electrosurgical system of claim 11, wherein the handle portion comprises a rotation knob coupled to a proximal end of the elongate actuator member.

15. The electrosurgical system of claim 11, wherein the distal portion of the distal jaw member comprises a hook member.

16. The electrosurgical system of claim 11, comprising an intermediate portion located between the distal portion and the proximal portion of the distal jaw member, wherein the first aperture is defined between the distal portion and the intermediate portion of the distal jaw member and a second aperture is defined between the intermediate portion and the proximal portion of the distal jaw member.

17. The electrosurgical system of claim 11, wherein the proximal jaw member comprises an electrically conductive sleeve defining an opening therethrough.

18. The electrosurgical system of claim 17, comprising an electrically insulative sleeve located within an opening defined by the conductive sleeve.

19. The electrosurgical system of claim 17, comprising an electrically insulative bushing fixedly coupled to the distal end of the elongate actuator member and located adjacent to the proximal portion of the distal jaw member.

20. A method of preparing an instrument for surgery, comprising:
obtaining the apparatus of claim 1;
sterilizing the surgical instrument; and
storing the surgical instrument in a sterile container.
